(19) United States
(12) Patent Application Publication
(10) Pub. No.: US 2002/0128455 A1
(43) Pub. Date: Sep. 12, 2002

(54) Growth factors, nucleic acid encoding them and methods for identification

(76) Inventors: Carlos Ibanez, Stockholm (SE); Henrik Jornvall, Stockholm (SE); Peter Lonnerberg, Bromma (SE)

Correspondence Address: DINSMORE & SHOHL, LLP, 1900 CHEMED CENTER, 255 EAST FIFTH STREET, CINCINNATI, OH, 45202, US

(21) Appl. No.: 09/886,426

(22) Filed: Jun. 21, 2001

Related U.S. Application Data
(60) Provisional application No. 60/216,078, filed on Jul. 6, 2000.

(30) Foreign Application Priority Data
Jun. 22, 2000 (SE) 002364-8

(51) Int. Cl. C07H 21/02; C07H 21/04

(52) U.S. Cl. 536/23.1

(57) ABSTRACT

The present invention relates to new polypeptide growth factors, designated MDF451 and MDF628, of the transforming growth factor-β (TGF-β) superfamily. The invention also relates to nucleic acid molecules encoding the said MDF451 and MDF628 polypeptides, as well as to methods for identification of agents mimicking or modulating the effects of growth factors of the TGF-β superfamily.

### TECHNICAL FIELD

The present invention relates to new polypeptide growth factors of the transforming growth factor-β (TGF-β) superfamily. The invention also relates to nucleic acid molecules encoding the said polypeptide growth factors, as well as to methods for identification of agents mimicking or modulating the effects of growth factors of the TGF-β superfamily.

### BACKGROUND ART

The transforming growth factor β (TGF-β) superfamily constitutes the largest group of polypeptide growth factors known, including the TGF-βs, activins, bone morphogenetic proteins (BMPs), growth and differentiation factors (GDFs), and neurotrophic factors of the GDNF ligand subfamily. Members of the TGF-β superfamily exhibit an extensive array of biological activities, regulating proliferation, lineage determination, differentiation, migration, adhesion and apoptosis during development, homeostasis and repair in practically all tissues, from flies to humans.

TGF-β superfamily members are characterized by a common three-dimensional (3D) fold containing a cysteine-knot (McDonald and Hendrickson, 1993). The pattern of cysteine residues is highly conserved in the primary sequence of different TGF-β superfamily members. So powerful is the structural constrain imposed by the conserved spacing of cysteine residues that even very distant members of the superfamily, such as TGF-β2 and GDNF, which are completely divergent in the sequence segments in between the cysteines (Lin et al., 1993), have an almost indistinguishable 3D structure (Eigenbrot and Gerber, 1997; Schlunegger and Grüter, 1992). approximately 55 kDa, and the type II receptors of approximately 70 kDa. Both receptors cooperate to ligand binding, type II receptors phosphorylate type I receptors, and the latter activate of the Smad family of signal transducers, which then translocate to the nucleus where they take part in a number of DNA binding complexes (for recent reviews, see e.g. Attisano and Wrana, 2000; Massagué and Chen, 2000; ten Dijke et al., 2000; Wrana, 2000). An exception to the scheme presented above is the glial cell line-derived neurotrophic factor (GDNF) ligand subfamily, which utilizes a completely unrelated receptor system, composed of a GPI-anchor ligand-binding subunit, the GFRα receptor, and a signaling subunit, the receptor tyrosine kinase c-Ret (Airaksinen et al., 1999).

There is a need for identification of new growth factors of the TGF-β superfamily, said growth factors representing signaling molecules potentially important for the control of cell survival, differentiation, proliferation or fate.

### BRIEF DESCRIPTION OF THE DRAWINGS

FIG. 1 Alignment of the amino acid sequences for MDF451 and MDF628 with the corresponding region of mouse GDNF and TGF-β2. Stars (*) indicate positions for conserved cysteine residues.

FIG. 2 Phylogenetic tree based on sequences of representative members of different TGF-β subfamilies, including MDF451 and MDF628.

FIG. 3 Expression of MDF451 and MDF628 mRNA in human tissues.

### DISCLOSURE OF THE INVENTION

Two novel genes, designated MDF451 and MDF628 respectively, have been identified. The new genes display sequence similarities to members of the TGF-β superfamily, primarily in the pattern of six conserved cysteine residues. MDF451 and MDF628 have no match in EST and GenBank databases, indicating that these genes may be expressed at low levels or restricted to a specific developmental stage or tissue. Phylogenetic tree analyses of these sequences together with representative members of different subfamilies of TGF-β molecules revealed that MDF451 and MDF628 constitute a novel and distinct subgroup within the TGF-β superfamily that appear to be more closely related to the GDNF subfamily of ligands. The two new members of the TGF-β superfamily are likely to represent new signaling molecules important for the control of cell survival, differentiation, proliferation or fate, in particular in the central nervous system.

Consequently, in a first aspect this invention relates to isolated nucleic acid molecules selected from:

(a) nucleic acid molecules comprising the nucleotide sequences set forth in SEQ ID NO: 1 and 3, respectively;

(b) nucleic acid molecules comprising a nucleotide sequence capable of hybridizing, under stringent hybridization conditions, to nucleotide sequences complementary the polypeptide coding region of a nucleic acid molecule as defined in (a) and which codes for MDF451 or MDFR628 polypeptide, or modified forms thereof; and

(c) nucleic acid molecules comprising a nucleic acid sequence which is degenerate as a result of the genetic code to a nucleotide sequence as defined in (a) or (b) and which codes for an MDF451 or MDF628 polypeptide, or modified forms thereof.

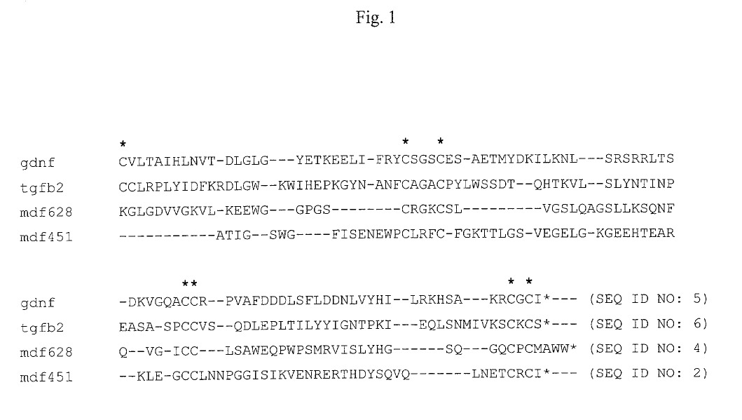

As used herein the phrase “modified forms” is intended to encompass polypeptides having substantially the same structural features and/or biological activities as the MDF451 or MDF628 polypeptide. The phrase “structural features” is in particular referring to the pattern of six conserved cysteine residues illustrated in FIG. 1 and by the formula

-Cys-(26)-Cys-(3)-Cys-(28)-Cys-Cys-(28)-Cys-(1)-Cys-

wherein the figures within brackets represent the approximate number of amino acid residues between the conserved cysteine residues. The phrase “biological activities” is intended to mean the ability to mediate the biological function characteristic for known members of the TGF-β superfamily (Massagué & Chen, 2000; ten Dijke et al., 2000; Wrana, 2000; Attisano & Wrana, 2000).

The term “stringent hybridization conditions” is known in the art from standard protocols (e.g. Ausubel et al., supra) and could be understood as e.g. hybridization to filter-bound DNA in 0.5 M NaHPO₄, 7% sodium dodecyl sulfate (SDS), 1 mM EDTA at +65° C., and washing in 0.1×SSC/0.1% SDS at +68° C.

It will thus be understood that the invention encompasses DNA molecules carrying modifications like substitutions, small deletions, insertions or inversions, which nevertheless encode polypeptides having substantially the structural features and/or the biological activity of the MDF451 or MDF628 polypeptides. Included in the invention are consequently DNA molecules, the nucleotide sequences of which are at least 90% homologous, preferably at least 95% homologous, with the nucleotide sequence set forth as SEQ ID NO: 1 or 3 in the Sequence Listing.

Included in the invention are also DNA molecule which nucleotide sequences are degenerate, because of the genetic code, to the nucleotide sequence shown as SEQ ID NO: 1 or 3. A sequential grouping of three nucleotides, a “codon”, codes for one amino acid. Since there are 64 possible codons, but only 20 natural amino acids, most amino acids are coded for by more than one codon. This natural “degeneracy”, or “redundancy”, of the genetic code is well known in the art. It will thus be appreciated that the DNA sequences shown in the Sequence Listing are only examples within a large but definite group of DNA sequences that will encode the MDF451 or MDF628 polypeptides.

In another important aspect, the invention provides isolated polypeptides encoded by the nucleic acid molecules defined above. In preferred forms of the invention, the said polypeptides have amino acid sequences set forth as SEQ ID NO: 2 and 4, respectively, of the Sequence Listing. However, the polypeptides according to the invention are not to be limited strictly to polypeptides having amino acid sequences identical with SEQ ID NO: 2 or 4. Rather the invention encompasses polypeptides carrying modifications like substitutions, small deletions, insertions or inversions, which polypeptides nevertheless have substantially the structural features and/or biological activity of the MDF451 or MDF628 polypeptides. Consequently, the invention also embraces polypeptides that have at least 99%, at least 95%, at least 90%, at least 85%, at least 80%, at least 75%, or at least 70% identity and/or homology to the polypeptides having amino acid sequences set forth as SEQ ID NO: 2 and 4, respectively.

In another aspect, the invention provides a vector harboring a nucleic acid molecule according to the invention. The said vector can e.g. be a replicable expression vector, which carries and is capable of mediating the expression of a nucleic acid molecule according to the invention. In the present context the term “replicable” means that the vector is able to replicate in a given type of host cell into which is has been introduced. Examples of vectors are viruses such as bacteriophages, cosmids, plasmids and other recombination vectors. Nucleic acid molecules are inserted into vector genomes by methods well known in the art.

Included in the invention is also a cultured host cell harboring a vector according to the invention. Such a host cell can be a prokaryotic cell, a unicellular eukaryotic cell or a cell derived from a multicellular organism. The host cell can thus e.g. be a bacterial cell such as an E. coli cell, a yeast cell, or a mammalian cell. The methods employed to effect introduction of the vector into the host cell are standard methods well known to a person familiar with recombinant DNA methods.

A further aspect of the invention is a process for production of a polypeptide, specifically an MDF451 or MDF628 polypeptide, which process comprises culturing a host cell as defined above under conditions whereby said polypeptide is produced, and recovering said polypeptide.

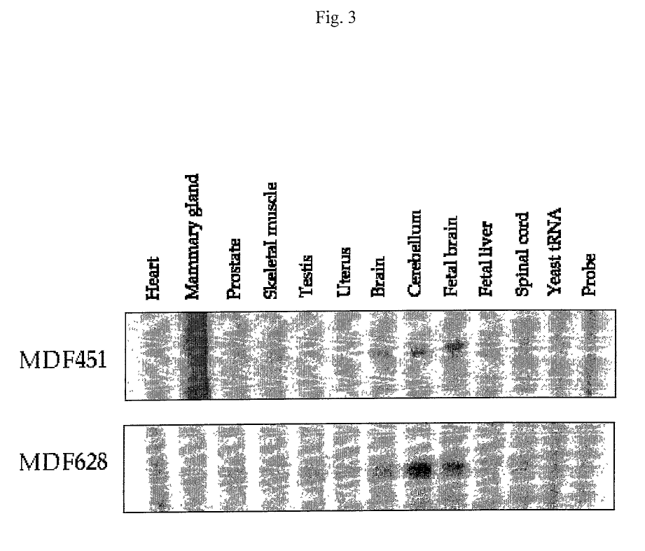

Expression of MDF451 and MDF628 in the brain (cf. Example 3 and FIG. 3) provides an indication that MDF polypeptides, as well as agents mimicking or modulating MDF activity, have utility for treating neurological disorders, such as schizophrenia, affective disorders, ADHD/ADD (i.e. Attention Deficit-Hyperactivity Disorder/Attention Deficit Disorder), and neural disorders such as Alzheimer's disease, Parkinson's disease, migraine, and senile dementia. Some other diseases for which MDF polypeptides, or agents mimicking or modulating MDF activity, may have utility include depression, anxiety, bipolar disease, epilepsy, neuritis, neurasthenia, neuropathy, metabolic diseases like diabetes type 2, obesity, neuroendocrine disorders such as growth hormone deficiency, inflammatory disorders, cancers, and the like.

As used herein, the phrase “MDF polypeptides” is intended to include MDF451 and MDF628, as well as modified forms thereof having essentially similar biological activities. The phrase “MDF activity” is intended to mean the biological activities of MDF polypeptides, such as the biological activities characteristic for known members of the TGFβ superfamily, which activities can mediate effects comprising neurotrophic, cell proliferation, tissue repair, wound healing, trauma treatment, cartilage inducing, bone inducing, connective tissue deposition, anti-inflammatory, lymphoid cell proliferation inhibition, hematopoietic, lymphopoietic, immunosuppressive, immunoregulatory, or epidermal cell proliferation inhibition effects.

Consequently, the invention includes a pharmaceutical composition comprising an MDF polypeptide and a pharmacologically acceptable carrier. The invention also includes a method for the treatment of disorders such as e.g. neurological disorders, comprising administering to a patient in need thereof an effective amount of an MDF polypeptide. As used herein, the term “treatment” is intended to include prophylaxis or attenuation of an existing condition.

The invention further comprises a method of mimicking the effects, such as e.g. neurological effects, in particular effects relating to neuron survival and neurite outgrowth, of TGF-β superfamily ligands in a mammalian subject, comprising administering to said subject an effective amount of the polypeptide according to any one of claims 3 to 6.

In yet a further aspect, the invention provides a diagnostic method comprising determining the amounts of an MDF polypeptide, or alternatively, a nucleic acid molecule encoding an MDF polypeptide, in a tissue or fluid sample from a human patient. The said diagnostic method is useful for diagnosis of medical conditions relating to effects characteristic for known members of the TGF-β superfamily.

The MDF polypeptides of the present invention may also be used to raise polyclonal or monoclonal antibodies. Such antibodies may be prepared by conventional techniques; see e.g. Antibodies: A Laboratory Manual, Harlow and Land (eds.), Cold Spring Harbor Laboratory Press, Cold Spring Harbor, N.Y., (1988); Monoclonal Antibodies, Hybridomas: A New Dimension in Biological Analyses, Kennet et al. (eds.), Plenum Press, New York (1980).

Antibodies of the invention are useful for e.g. therapeutic purposes (by modulating activity of MDF451 or MDF628), diagnostic purposes to detect or quantitate MDF451 or MDF628, and purification of MDF451 or MDF628. Kits comprising an antibody of the invention for any of the purposes described herein are also included in the invention. In general, a kit of the invention also includes a control antigen for which the antibody is immunospecific.

In a further aspect, the MDF polypeptides according to the invention can be utilized in methods for identification of agents mimicking or modulating the effects, such as neurological effects, of TGF-β superfamily ligands. As used herein, the term “agent” means a biological or chemical compound such as a simple or complex organic molecule, a peptide, a protein or an oligonucleotide.

Consequently, the invention provides a method for the identification of an agent mimicking the effects of TGFβ superfamily ligands, comprising

(i) contacting a test agent with a mammalian cell;

(ii) comparing the effect of the said test agent on the said mammalian cell with the effect of an MDF polypeptide according to the invention, whereby a similar activity indicates that said test agent is mimicking the effects of TGF-β superfamily ligands.

Also included in the invention is a method for identifying an agent useful for decreasing or inhibiting the biological activities of an MDF polypeptide, or decreasing or inhibiting the expression of a nucleic acid molecule encoding an MDF polypeptide, said method comprising the steps

(i) contacting a test agent with an MDF polypeptide or with a nucleic acid molecule encoding an MDF polypeptide; and

(ii) determining whether said test agent decreases or inhibits the biological activities of the said MDF polypeptide, or decreases or inhibits the expression of the said nucleic acid molecule.

Additional features of the invention will be apparent from the following Examples. Examples 1 to 3 are actual, while the remaining Examples are prophetic.

Throughout this description the terms “standard methods” and “standard procedures”, when used in the context of molecular biology techniques, are to be understood as protocols and procedures found in an ordinary laboratory manual such as: Current Protocols in Molecular Biology, editors F. Ausubel et al., John Wiley and Sons, Inc. 1994, or Sambrook, J., Fritsch, E. F. and Maniatis, T., Molecular Cloning: A laboratory manual, 2^{nd} Ed., Cold Spring Harbor Laboratory Press, Cold Spring Harbor, N.Y. 1989.

### EXAMPLES

### Examples 1

### Identification of MDF451 and MDF628

The robustness of the cysteine pattern of TGF-β superfamily members was utilized in order to identify novel members of this family. A search engine called Motifer (Jörnvall, 1999) was used. Motifer is a software tool able to find directly in nucleotide databases very distant homologues to an amino acid query sequence.

The following query sequence, derived from the primary sequences of members of the GDNF ligand family, was used:

-Cys-(26)-Cys-(3)-Cys-(28)-Cys-Cys-(28)-Cys-(1)-Cys-

The figures within brackets represent the number of amino acid residues between the conserved cysteine residues.

The publicly available database of the Human Genome Project (available via http://www.ncbi.nlm.nih.gov) was searched using Motifer and the above query sequence. Sequences of known members of the TGF-β superfamily were included in the search. The first 300 positions of the obtained output file corresponded to previously described genes of the TGF-β superfamily. In addition, two novel sequences were identified displaying conservation of all but the first Cys of the query. The two sequences were provisionally named MDF (Motifer-Derived Factor) 451 (SEQ ID NOs: 1 and 2) and MDF 628 (SEQ ID NOs: 3 and 4), respectively. Neither MDF451 nor MDF628 could be identified applying other algorithms, including BLAST, to the same database. The two identified sequences, aligned to the corresponding region of mouse GDNF (SEQ ID NO: 5; positions 147 through 240 of GenBank accession number AAB 18672) and mouse TGF-β2 (SEQ ID NO: 6; positions 317 through 414 of GenBank accession number CAA40672), are shown in FIG. 1.

The absence of the first Cys residue in the two sequences could be explained by the presence of an intron somewhere upstream of the sequence coding for the first Cys residue. In most other members of the TGF-β superfamily, including GDNF subfamily ligands, an intron is located upstream of the first Cys residue and the prohormone proteolytic cleavage site, so that the complete mature sequence is encoded in a single exon. The position of an intron in the mature sequence, if confirmed, suggest a novel gene structure for the MDF451 and MDF628 genes.

No stop codons could be found within the predicted coding sequences of MDF451 or MDF628. The genes contain a stop codon after 1 and 3 amino acid residues, respectively, downstream of the last cysteine, a feature that is highly conserved in all members of the TGF-β superfamily. No other cysteine in addition to those conforming to the query pattern could be found in either MDF451 or MDF628. This fact, together with the high conservation of the observed Cys pattern and the lack of internal stop codons, suggest that MDF451 and MDF628 are not pseudogenes.

### Example 2

### Sequence Alignment

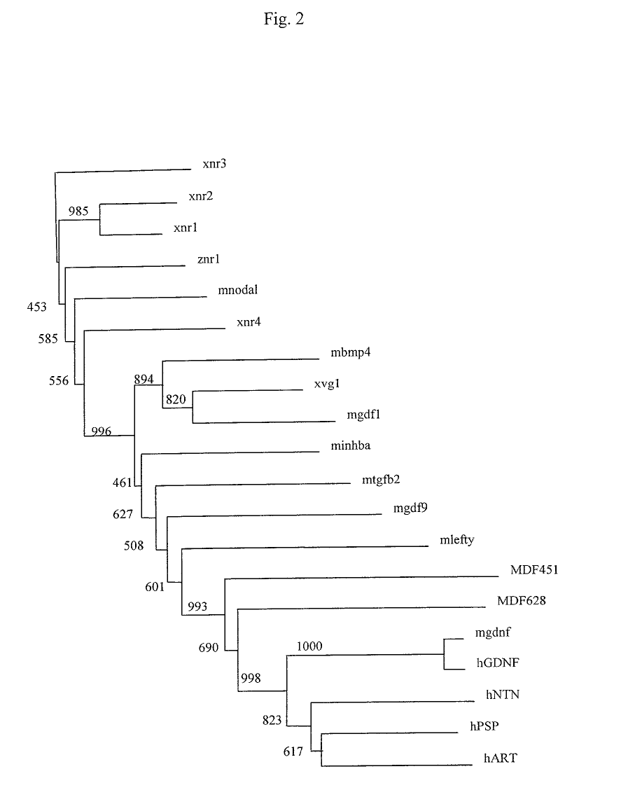

An alignment of sequences of representative members of different TGF-β subfamilies including MDF451 and MDF628 was made using the multiple alignment program ClustalX, which is available from the Center for Scientific Computing (http://www.csc.fi; see also Thompson et al., 1997). A phylogenetic tree, constructed with ClustalX, is shown in FIG. 2. Bootstrap numbers are indicated in the nodes of the tree, and give an estimate of the probability that the corresponding node represent to a true distinct branch in the tree. Nodes with a low bootstrap value are not well supported by the data and suggest that alternative relationships may also be possible. In this case, the two MDFs appear in a distinct branch of the tree leading to the GDNF ligand subfamily. This branch has the relatively high bootstrap value of 993, indicating that it is well supported by the data and suggesting that MDF451 and MDF628 may represent distant relatives of GDNF subfamily ligands.

### Example 3

### Expression of MDF451 and MDF628 mRNA in Human Tissues

Expression of MDF451 and MDF628 mRNA was assayed by RNase protection assay according to standard procedures. Human RNAs were purchased from Clontech. For RNase protection assays, PCR fragments of MDF451 and MDF628 were subcloned into pBSKS+, linearized, and used as template for T7 RNA polymerase using a kit from

Promega. 10 μg of total RNA was hybridized to [(α-³²P]CTP-labeled cRNA probes using a kit from Ambion according to the manufacturer's instructions. Protected bands were visualized and quantified using a STORM840 phosphorimager and ImageQuant software (Molecular Dynamics).

From the samples included in this survey, MDF451 and MDF628 appear to be predominantly expressed in nervous tissue, including fetal and adult brain and cerebellum (FIG. 3). Weak expression of MDF451 can also be seen in skeletal muscle and spinal cord and of MDF628 in testis. These data argues strongly that both MDFs are indeed expressed genes, and indicate that they may represent neuron survival or differentiation factors.

### Example 4

### Cloning of Full Length Human MDF451 and MDF628

Full length clones for human MDF451 and MDF628 are obtained by Rapid Amplification of cDNA Ends (RACE) using human cerebellum cDNA (Clontech) and specific upstream primers according to standard procedures and manufacturers' instructions. The extended products obtained from RACE are verified by automatic DNA sequencing and ligated to the partial MDF fragments to obtain full-length clones.

### Example 5

### Expression of Recombinant MDF Protein

The full-length open reading frames (ORFs) of MDF451 and MDF628 are subcloned in the mammalian expression vector pcDNA3 for transient expression in COS cells according to standard procedures. An epitope tag is introduced at the time of cloning to monitor protein during purification stages. MDF protein is harvested from COS cell supernatants and purified using conventional chromatography techniques, including ion exchange, size-exclusion and reverse phase chromatography (RPC). Purification is monitored by Western blotting using antibodies specific to the tagged MDF proteins. Final purity is assessed by silver staining of SDS/PAGE gels.

### Example 6

### Cloning of Rat and Mouse Homologues of Human MDF451 and MDF628

Using probes from human MDF451 and MDF628, phage libraries of rat and mouse brain are screened by low hybridization procedures according to standard procedures. cDNAs corresponding to rodent MDFs are isolated, sequenced and extended by RACE to obtain full-length clones as needed.

### Example 7

### Anti-MDF Antibodies

Standard techniques are employed to generate polyclonal or monoclonal antibodies to MDF451 and MDF628, and to generate useful antigen-binding fragments thereof or variants thereof, including “humanized” variants. Such protocols can be found, for example, in Sambrook et al. (1989) and Harlow et al. (Eds.), Antibodies, A Laboratory Manual; Cold Spring Harbor Laboratory; Cold Spring Harbor, N.Y. (1988).

Peptides obtained from hydrophilic regions of the mouse and rat MDF451 and MDF628 sequences can be synthesized, conjugated to Keyhole Limpet Hemocyanin (KLH) and used to immunize rabbits for antisera preparation. Titer of the subsequent boostings can be monitored by Western blotting using purified recombinant MDF protein.

### Example 8

### In situ Hybridization

Expression of MDFs mRNA in developing mouse and rat embryos is studied by in situ hybridization according to standard methods. Briefly, MDFs riboprobes are labeled with ³⁵S-UTP or ³³P-UTP using linearized template DNA fragments and reagents for in vitro transcription from Promega. For in situ hybridization, 14 mm sections are thawed onto 3-α aminopropyl ethoxysilane coated slides for hybridization with radiolabeled probes as follows. Following fixation in 4% paraformaldehyde for 15 min, slides are rinsed once in PBS and twice in distilled water. Tissue is de-proteinated in 0.2 M HCl for 10 min, acetylated with 0.25% acetic anhydride in 0.1M ethanolamine for twenty minutes and dehydrated with increasing concentrations of ethanol. Slides are incubated 16 h in a humidified chamber at +58° C. with 8×10⁵ cpm of probe in 300 ml of hybridization cocktail (50% formamide, 20 mM Tris-HCl (pH 7.6), 1 mM EDTA pH 8.0, 0.3 M NaCl, 0.1 M dithiothreitol, 0.5 mg/ml yeast tRNA, 0.1 mg/ml poly A-RNA, 1×Denhardt's solution and 10% dextran sulphate). Slides are first washed at room temperature in Formamide:SSC (1:1) followed by 30 min at +65° C. in 1×SSC. Single-stranded RNA is digested by RNAse treatment (10 mg/ml) for 30 min at +37° C. in 0.5 M NaCl, 20 mM Tris-HCl (pH 7.5), 2mM EDTA. Tissue is washed twice with 1×SSC at +65° C. for 30 min before dehydration in ethanol and air drying. Slides are either exposed to β-max x-ray film (Amersham, UK) for 10 to 20 days, or dipped in NTB-2 photoemulsion diluted 1:1 in water (Eastman-Kodak) exposed at +4° C. for 3-5 weeks, developed with D19 (Eastman-Kodak Co.), fixed with Al-4 (Agfa Gevaert) and counterstained with cresyl violet.

### REFERENCES

Airaksinen, M. S., Titievsky, A., and Saarma, M. (1999). GDNF family neurotrophic factor signaling: Four masters, one servant? Mol Cell Neurosci 13, 313-325.

Attisano, L. and Wrana, J. L. (2000). Smads as transcriptional co-modulators. Current Opinion in Cell Biology 12, 235-243.

Eigenbrot, C., and Gerber, N. (1997). X-ray structure of glial cell-derived neurotrophic factor at 1.9 angstrom resolution and implications for receptor binding. Nature Struct. Biol. 4, 435-438.

Jörnvall, H. (1999). Motifer, a search tool for finding amino acid sequence patterns from nucleotide sequence databases. FEBS Lett 456, 85-88.

Lin, L.-F. H., Doherty, D., Lile, J., Bektesh, S., and Collins, F. (1993). GDNF: a glial cell line-derived neurotrophic factor for midbrain dopaminergic neruons. Science 260, 1130-1132.

Massagué, J., and Chen, Y.-G. (2000). Controlling TGF-β signaling. Genes & Dev 14, 627-644.

McDonald, N. Q., and Hendrickson, W. A. (1993). A structural superfamily of growth factors containing a cysteine knot motif. Cell 73, 421-424.

Schlunegger, M., and Grüter, M. (1992). An unusual feature revealed by the crystal structure at 2.2 Å resolution of human transforming growth factor-β2. Nature 358, 430-434.

ten Dijke, P., Miyazono, K., and Heldin, C. H. (2000). Signaling inputs converge on nuclear effecters in TGF-beta signaling [Review]. Trends in Biochemical Sciences 25, 64-70.

Thompson, J. D. et al. (1997). The ClustalX windows interface: flexible strategies for multiple sequence alignment aided by quality analysis tools. Nucleic Acid Research 24, 4876-4882.

Wrana, J. (2000). Regulation of Smad activity [Review]. Cell 100, 189-192

1. An isolated nucleic acid molecule selected from:
(a) nucleic acid molecules comprising a nucleotide sequence as shown in SEQ ID NO: 1;
(b) nucleic acid molecules comprising a nucleotide sequence capable of hybridizing, under stringent hybridization conditions, to a nucleotide sequence complementary the polypeptide coding region of a nucleic acid molecule as defined in (a) and which codes for an MDF451 polypeptide or a modified form thereof; and
(c) nucleic acid molecules comprising a nucleic acid sequence which is degenerate as a result of the genetic code to a nucleotide sequence as defined in (a) or (b) and which codes for an MDF451 polypeptide or a modified form thereof.

2. An isolated nucleic acid molecule selected from:
(a) nucleic acid molecules comprising a nucleotide sequence as shown in SEQ ID NO: 3;
(b) nucleic acid molecules comprising a nucleotide sequence capable of hybridizing, under stringent hybridization conditions, to a nucleotide sequence complementary the polypeptide coding region of a nucleic acid molecule as defined in (a) and which codes for an MDF628 polypeptide or a modified form thereof; and
(c) nucleic acid molecules comprising a nucleic acid sequence which is degenerate as a result of the genetic code to a nucleotide sequence as defined in (a) or (b) and which codes for an MDF628 polypeptide or a modified form thereof.

3. An isolated polypeptide encoded by the nucleic acid according to claim 1.

4. The isolated polypeptide according to claim 3 having the amino acid sequence shown as SEQ ID NO: 2 in the Sequence Listing

5. An isolated polypeptide encoded by the nucleic acid according to claim 2.

6. The isolated polypeptide according to claim 5 having the amino acid sequence shown as SEQ ID NO: 4 in the Sequence Listing

7. A vector harboring the nucleic acid molecule according to claim 1 or 2.

8. A cultured host cell harboring a vector according to claim 7.

9. A process for production of a polypeptide, comprising culturing a host cell according to claim 8 under conditions whereby said polypeptide is produced, and recovering said polypeptide.

10. A pharmaceutical composition comprising the polypeptide according to any one of claims 3 to 6 and a pharmacologically acceptable carrier.

11. The pharmaceutical composition according to claim 10 for use in the treatment of neurological disorders.

12. Use of a polypeptide according to any one of claims 3 to 6 in the manufacture of a medicament for the treatment of neurological disorders.

13. A method for the treatment of neurological disorders, comprising administering to a patient in need thereof an effective amount of a polypeptide according to any one of claims 3 to 6.

14. A method of mimicking the effects of TGF-β superfamily ligands in a mammalian subject, comprising administering to said subject an effective amount of the polypeptide according to any one of claims 3 to 6.

15. The method according to claim 14 wherein the said effects are neurological effects.

16. The method according to claim 15 wherein the said neurological effects comprise effects relating to neuron survival and neurite outgrowth.

17. A diagnostic method comprising determining the amounts of the polypeptide according to any one of claims 3 to 6 in a tissue or fluid sample from a human patient.

18. A diagnostic method comprising determining the amounts of the nucleic acid molecule according to claim 1 or 2 in a tissue or fluid sample from a human patient.

19. An antibody capable of binding to a polypeptide according to any one of claims 3 to 6.

20. The use of a polypeptide according to any one of claims 3 to 6 in a method for identification of an agent mimicking the effects of TGF-β superfamily ligands.

21. The use according to claim 20 wherein the said effects are neurological effects.

22. A method for identifying an agent mimicking the effects of TGF-β superfamily ligands, comprising
(i) contacting a test agent with a mammalian cell;
(ii) comparing the effect of the said test agent on the said mammalian cell with the effect of a polypeptide according to any one of claims 3 to 6, whereby a similar activity indicates that said test agent is mimicking the effects of TGF-β superfamily ligands.

23. A method for identifying an agent useful for decreasing or inhibiting the expression of an MDF451 or MDF628 nucleic acid molecule, said method comprising the steps
(i) contacting a test agent with an MDF451 or MDF628 nucleic acid molecule according to claim 1 or 2; and
(ii) determining whether said test agent decreases or inhibits the expression of the said MDF451 or MDF628 nucleic acid molecule.

24. A method for identifying an agent useful for decreasing or inhibiting the biological activities of an MDF451 or MDF628 polypeptide, said method comprising the steps
(i) contacting a test agent with an MDF451 or MDF628 polypeptide according to any one of claim 3 to 6; and
(ii) determining whether said test agent decreases or inhibits the biological activities of the said MDF451 or MDF628 polypeptide.


SEQUENCE LISTING (6 sequences)

SEQ ID NO: 1 - DNA, 246 bp, Homo sapiens
  CDS (1)..(243)
gccaccattgggagttggggcttcatatcagaaaatgagtggccatgtctacggttctgctttgggaagaccactctgggcagtgtggagggggagcttgggaaaggagaggaacatacagaggccaggaagctggagggatgctgtctcaataatccaggtgggatatcaattaaggtggagaacagggagaggactcatgactattcccaagttcaactcaatgagacttgccgatgcatttga

SEQ ID NO: 2 - protein, 81 aa, Homo sapiens
ATIGSWGFISENEWPCLRFCFGKTTLGSVEGELGKGEEHTEARKLEGCCLNNPGGISIKVENRERTHDYSQVQLNETCRCI

SEQ ID NO: 3 - DNA, 237 bp, Homo sapiens
  CDS (1)..(234)
aagggcctgggggatgttgtggggaaggtcctgaaggaggaatggggagggccaggatcctgcaggggcaagtgcagtctggttggatctctccaggcagggagcctcttaaaatcccagaatttccaggtgggcatctgctgcctttcggcatgggagcagccatggccatcaatgagggtaatttccttgtatcatgggtcccagggacagtgtccctgcatggcctggtggtga

SEQ ID NO: 4 - protein, 78 aa, Homo sapiens
KGLGDVVGKVLKEEWGGPGSCRGKCSLVGSLQAGSLLKSQNFQVGICCLSAWEQPWPSMRVISLYHGSQGQCPCMAWW

SEQ ID NO: 5 - protein, 94 aa, Mus musculus
CVLTAIHLNVTDLGLGYETKEELIFRYCSGSCESAETMYDKILKNLSRSRRLTSDKVGQACCRPVAFDDDLSFLDDNLVYHILRKHSAKRCGCI

SEQ ID NO: 6 - protein, 98 aa, Mus musculus
CCLRPLYIDFKRDLGWKWIHEPKGYNANFCAGACPYLWSSDTQHTKVLSLYNTINPEASASPCCVSQDLEPLTILYYIGNTPKIEQLSNMIVKSCKCS